(19) United States
(12) Patent Application Publication
(10) Pub. No.: US 2009/0124656 A1
(43) Pub. Date: May 14, 2009

(54) Heterocyclic Oxime Compounds a Process for Their Preparation and Pharmaceutical Compositions Containing Them

(75) Inventors: Cicile Parmenon, Tours (FR); Marie-Claude Viaud-Massuard, Tours (FR); Jerome Guillard, Liguell (FR); Catherine Dacuet, Villeneuve D' Ascq (FR); Alain Ktorza, Nogent-Sur-Marne (FR); Daniel- Henri Caignard, Boisemont (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: LES LABORATOIRES SERVIER, COURBEVOIE CEDEX (FR)

(21) Appl. No.: 11/883,107

(22) PCT Filed: Jan. 26, 2006
(86) PCT No.: PCT/FR2006/000174

§ 371 Date: Jul. 25, 2007

(30) Foreign Application Priority Data
Jan. 27, 2005 (FR) 0500842

(51) Int. Cl. A61K 31/47

(52) U.S. Cl. 514/311; 546/165

(57) ABSTRACT

Compounds of formula (I):

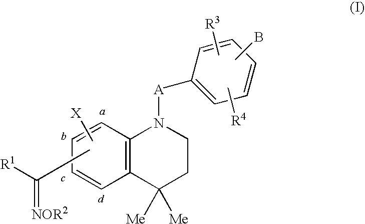

wherein:
R¹, R², R³, R⁴ and X are as defined in the description,
A represents an alkylene chain as defined in the description,
B represents an alkyl or alkenyl group substituted by a group

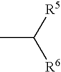

or R⁷, or B represents a group

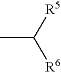

or R⁷.

Medicinal products containing the same which are useful as hypoglycaemic and hypolipaemic agents.

The present invention relates to new heterocyclic oxime compounds, to a process for their preparation and to pharmaceutical compositions containing them.

The compounds described in the present invention are new and have pharmacological properties that are of special interest: they are excellent hypoglycaemic and hypolipaemic agents.

The treatment of non-insulin-dependent type II diabetes remains unsatisfactory despite the introduction onto the market of a large number of oral hypoglycaemic compounds intended to facilitate the secretion of insulin and to promote its action in peripheral target tissues.

During the last ten years, a class of compounds having a thiazolidinedione structure (U.S. Pat. No. 5,089,514, U.S. Pat. No. 5,306,726) has demonstrated a marked anti-diabetic activity by promoting sensitivity to insulin in target peripheral tissues (skeletal muscle, liver, adipose tissue) of animal models having non-insulin-dependent type II diabetes. Those compounds also lower the levels of insulin and of lipids in the same animal models and induce in vitro differentiation of preadipocyte cell lines into adipocyte cell lines (A. Hiragun et al., J. Cell. Physiol., 1988, 134, 124-130; R. F. Kleitzen et al., Mol. Pharmacol., 1992, 41, 393-398). The treatment of preadipocyte cell lines with the thiazolidinedione rosiglitazone brings about inducement of the expression of specific genes of the lipid metabolism, such as aP2 and adipsin, and also the expression of the glucose transporters GLUT1 and GLUT4, suggesting that the effect of the thiazolidinediones observed in vivo may be mediated via adipose tissue. That specific effect is obtained by the stimulation of nuclear transcription factors: <<peroxisome proliferator-activated receptor gamma>> (PPAR γ2). Such compounds are capable of restoring sensitivity to insulin in peripheral tissues, such as adipose tissue or skeletal muscle (J. E. Gerich, New Engl. Med., 19, 321, 1231-1245).

Compounds having a thiazolidinedione structure (troglitazone, rosiglitazone) have demonstrated disturbing side effects in man, however, especially liver problems (Script No 2470, 1999, Sep. 8, 25).

A large number of hypoglycaemic agents have significant side effects (hepatic, cardiac, haematopoietic), which limit their long-term use in the treatment of non-insulin-dependent type II diabetes.

The development of new therapeutic agents that are less toxic and that are active over the long term is absolutely necessary in this pathology.

Moreover, hyperlipidaemia is often observed in diabetics (Diabetes Care, 1995, 18 (supplement 1), 86/8/93). The association of hyperglycaemia with hyperlipidaemia increases the risk of cardiovascular disease in diabetics. Hyperglycaemia, hyperlipidaemia and obesity have become pathologies of the modern world marked by the intake of food in large quantities and a chronic lack of exercise.

The increase in frequency of those pathologies calls for the development of new therapeutic agents that are active in such disorders: compounds having an excellent hypoglycaemic and hypolipaemic activity whilst avoiding the side effects observed with thiazolidinediones are consequently very beneficial in the treatment and/or prophylaxis of those pathologies, and are indicated especially in the treatment of non-insulin-dependent type II diabetes for reducing peripheral insulin resistance and for normalising glucose control.

In addition to the fact that they are new, the compounds of the present invention meet the above pharmacological criteria and are excellent hypoglycaemic and hypolipaemic agents.

The present invention relates more especially to the compounds of formula (I):

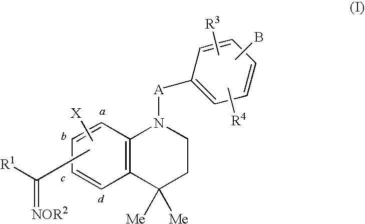

wherein
A represents a (C₁-C₆)alkylene chain in which a CH₂ group may be replaced by a hetero atom selected from oxygen and sulphur, or by an NRₐ group (wherein Rₐ represents a hydrogen atom or a linear or branched (C₁-C₆)alkyl group), or by a phenylene or naphthylene group,
R¹ and R², which may be identical or different, each represents a hydrogen atom, a linear or branched (C₁-C₆)alkyl group, a linear or branched (C₂-C₆)alkenyl group, a linear or branched (C₂-C₆)alkynyl group, an aryl group, an aryl-(C₁-C₆)alkyl group in which the alkyl moiety may be linear or branched, an aryl-(C₂-C₆)alkenyl group in which the alkenyl moiety may be linear or branched, an aryl-(C₂-C₆)alkynyl group in which the alkynyl moiety may be linear or branched, a heteroaryl group, a heteroaryl-(C₁-C₆)alkyl group in which the alkyl moiety may be linear or branched, a heteroaryl-(C₂-C₆)alkenyl group in which the alkenyl moiety may be linear or branched, a heteroaryl-(C₂-C₆)alkynyl group in which the alkynyl moiety may be linear or branched, a (C₃-C₈)cycloalkyl group, a (C₃-C₈)cycloalkyl-(C₁-C₆)alkyl group in which the alkyl moiety may be linear or branched or a linear or branched (C₁-C₆)poly-haloalkyl group,
R³ and R⁴, which may be identical or different, each represents a hydrogen atom, a halogen atom, or an R, OR or NRR′ group wherein R and R′, which may be identical or different, each represents a hydrogen atom or a linear or branched (C₁-C₆)alkyl group, a linear or branched (C₂-C₆)alkenyl group, a linear or branched (C₂-C₆)alkynyl group, an aryl group, an aryl-(C₁-C₆)alkyl group in which the alkyl moiety may be linear or branched, an aryl-(C₂-C₆)alkenyl group in which the alkenyl moiety may be linear or branched, an aryl-(C₂-C₆)alkynyl group in which the alkynyl moiety may be linear or branched, a heteroaryl group, a heteroaryl-(C₁-C₆)alkyl group in which the alkyl moiety may be linear or branched, a heteroaryl-(C₂-C₆)alkenyl group in which the alkenyl moiety may be linear or branched, a heteroaryl-(C₂-C₆)alkynyl group in which the alkynyl moiety may be linear or branched, a (C₃-C₈)cycloalkyl group, a (C₃-C₈)cycloalkyl-(C₁-C₆)alkyl group in which the alkyl moiety may be linear or branched or a linear or branched (C₁-C₆)polyhaloalkyl group,
or R³ and R⁴, together with the carbon atoms carrying them, when they are carried by two adjacent carbon atoms, form a ring that comprises 5 or 6 ring members and that may optionally contain a hetero atom selected from oxygen, sulphur and nitrogen,
X represents a hydrogen atom, a halogen atom, or a linear or branched (C₁-C₆)alkyl group,
B represents a linear or branched (C₁-C₆)alkyl group or a linear or branched (C₂-C₆)alkenyl group, those groups being substituted:
   by a group of formula (II):

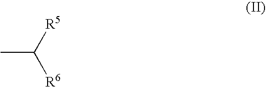

wherein:
   R⁵ represents a

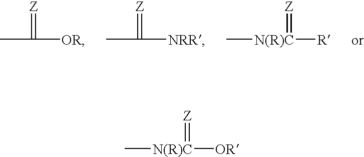

group wherein Z represents an oxygen atom or a sulphur atom and R and R′, which may be identical or different, are as defined hereinbefore,
and R⁶ represents an aryl group, an arylalkyl group in which the alkyl moiety contains from 1 to 6 carbon atoms and may be linear or branched, a heteroaryl group, a heteroarylalkyl group in which the alkyl moiety contains from 1 to 6 carbon atoms and may be linear or branched, CN, tetrazole

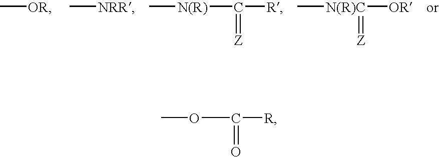

wherein Z is as defined hereinbefore and R and R′, which may be identical or different, may take the same meanings as defined hereinbefore,
or by a group R⁷, R⁷ representing a CN, tetrazole,

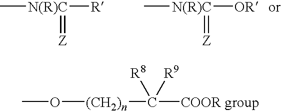

wherein Z is as defined hereinbefore and R and R′, which may be identical or different, may take the same meanings as defined hereinbefore, n represents 0, 1, 2, 3, 4, 5 or 6, and R⁸ and R⁹, which may be identical or different, each represents a hydrogen atom or a linear or branched (C₁-C₆)alkyl group, wherein R⁸ and R⁹ cannot simultaneously represent a hydrogen atom,
or B represents a group of formula (II) or a group R⁷ as defined hereinbefore,
   it being understood that:
the oxime R¹—C(═N—OR²)— may have the Z or E configuration,
aryl is understood to mean a phenyl, naphthyl or biphenyl group, wherein those groups may optionally be partially hydrogenated,
heteroaryl is understood to mean any aromatic mono- or bi-cyclic group containing from 5 to 10 ring members, which in the case of bicyclic heteroaryl groups may optionally be partially hydrogenated on one of the rings, and containing from 1 to 3 hetero atoms selected from oxygen, nitrogen and sulphur,
   wherein the aryl and heteroaryl groups so defined may optionally be substituted by from 1 to 3 identical or different groups selected from linear or branched (C₁-C₆)alkyl, linear or branched (C₁-C₆)polyhaloalkyl, linear or branched (C₁-C₆)alkoxy, hydroxy, carboxy, linear or branched (C₁-C₆)alkoxycarbonyl, linear or branched (C₁-C₆)acyloxy, formyl, linear or branched (C₁-C₆)acyl, aroyl, NR_{b}R_{c} (wherein R_{b} and R_{c}, which may be identical or different, each represents a hydrogen atom, a linear or branched (C₁-C₆)alkyl group, an aryl group or a heteroaryl group), amido, nitro, cyano, and halogen atoms,
   to their enantiomers and diastereoisomers, and also to pharmaceutically acceptable addition salts thereof with an acid or a base.

Amongst the pharmaceutically acceptable acids there may be mentioned, without implying any limitation, hydrochloric acid, hydrobromic acid, sulphuric acid, phosphonic acid, acetic acid, trifluoroacetic acid, lactic acid, pyruvic acid, malonic acid, succinic acid, glutaric acid, fumaric acid, tartaric acid, maleic acid, citric acid, ascorbic acid, methanesulphonic acid, camphoric acid, oxalic acid, etc. . . .

Amongst the pharmaceutically acceptable bases there may be mentioned, without implying any limitation, sodium hydroxide, potassium hydroxide, triethylamine, tert-butylamine, etc.

Preferably, the group R¹—C(═N—OR²)— is in the c position.

A preferred group for R³ and R⁴ is the hydrogen atom.

X advantageously represents a hydrogen atom or an alkyl group such as, for example, the methyl group.

Preferably, A represents an alkylene chain in which a CH₂ group may be replaced by a hetero atom and more especially by an oxygen atom.

The invention relates more especially to compounds of formula (I) wherein A represents an ethyleneoxy group.

Preferred R² groups are the hydrogen atom and alkyl groups, such as, for example, the methyl group.

R¹ advantageously represents a phenyl group that is unsubstituted or substituted by one or more substituents selected from groups such as alkyl, alkoxy, cyano, alkoxycarbonyl, carboxy, and halogen atoms.

Preferred groups B are alkyl or alkenyl groups, and more especially alkyl groups, substituted by a

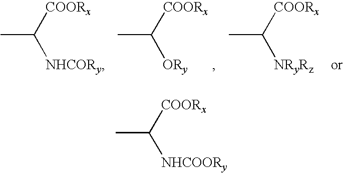

group,
wherein Rₓ, R_{y} and R_{z}, which may be identical or different, each represents a hydrogen atom or an alkyl group, such as, for example, a methyl, ethyl, propyl, isopropyl, butyl, isobutyl, tert-butyl, pentyl, isopentyl, neopentyl or hexyl group, a polyhaloalkyl group, such as, for example, a trifluoromethyl or trifluoroethyl group, or a phenyl group that is unsubstituted or substituted by an alkyl, polyhaloalkyl, acyl or aroyl group.

The invention relates more especially to compounds of formula (I) wherein B represents an alkyl or alkenyl group substituted by a

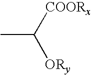

group wherein Rₓ and R_{y} are are as defined hereinbefore.

Likewise advantageously, B represents a group

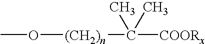

wherein n and Rₓ are as defined hereinbefore.

Even more especially, B represents a group

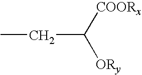

wherein Rₓ and R_{y} are as defined hereinbefore.

The invention relates very advantageously to the compounds of formula (I) wherein:

A represents a —CH₂—CH₂—O— chain,
R³ and R⁴ simultaneously represent a hydrogen atom,
R² represents a hydrogen atom or an alkyl group,
R¹ represents an unsubstituted phenyl group, and
B represents a group

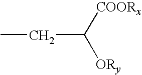

wherein Rₓ and R_{y} are as defined hereinbefore.
Even more especially, the invention relates to the following compounds of formula (I):
ethyl 2-ethoxy-3-{4-[2-(6-[(hydroxyimino)(phenyl)methyl]-4,4-dimethyl-3,4-dihydro-1(2H)-quinolinyl)ethoxy]phenyl}propanoate,
ethyl 2-ethoxy-3-{4-[2-(6-[(methoxyimino)(phenyl)methyl]-4,4-dimethyl-3,4-dihydro-1(2H)-quinolinyl)ethoxy]phenyl}propanoate,
2-ethoxy-3-{4-[2-(6-[(methoxyimino)(phenyl)methyl]-4,4-dimethyl-3,4-dihydro-1(2H)-quinolinyl)ethoxy]phenyl}propanoic acid,
ethyl (2S)-2-ethoxy-3-{(4-[2-(6-[(methoxyimino)(phenyl)methyl]-4,4-dimethyl-3,4-dihydro-1(2H)-quinolinyl)ethoxy]phenyl}propanoate,
(2S)-2-ethoxy-3-{4-[2-(6-[(methoxyimino)(phenyl)methyl]-4,4-dimethyl-3,4-dihydro-1(2H)-quinolinyl)ethoxy]phenyl}propanoic acid,
ethyl 3-{4-[2-(6-[(methoxyimino)(phenyl)methyl]-4,4-dimethyl-3,4-dihydro-1(2H)-quinolinyl)ethoxy]phenyl}-2-[4-(trifluoromethyl)phenoxy]propanoate,
2-(acetoxy)-3-{(4-[2-(6-[(methoxyimino)(phenyl)methyl]-4,4-dimethyl-3,4-dihydro-1(2H)-quinolinyl)ethoxy]phenyl}propanoic acid,
ethyl 3-{4-[2-(6-[(methoxyimino)(phenyl)methyl]-4,4-dimethyl-3,4-dihydro-1(2H)-quinolinyl)ethoxy]phenyl}-2-(2,2,2-trifluoroethoxy)propanoate,
3-{4-[2-(6-[(methoxyimino)(phenyl)methyl]-4,4-dimethyl-3,4-dihydro-1(2H)-quinolinyl)ethoxy]phenyl}-2-(2,2,2-trifluoroethoxy)propanoic acid,
ethyl 2-(2-benzoylanilino)-3-{4-[2-(6-[(methoxyimino)(phenyl)methyl]-4,4-dimethyl-3,4-dihydro-1(2H)-quinolinyl)ethoxy]phenyl}propanoate,
2-(2-benzoylanilino)-3-{4-[2-(6-[(methoxyimino)(phenyl)methyl]-4,4-dimethyl-3,4-dihydro-1(2H)-quinolinyl)ethoxy]phenyl}propanoic acid,
ethyl (2S)-3-{4-[2-(6-[(4-cyanophenyl)(methoxyimino)methyl]-4,4-dimethyl-3,4-dihydro-1(2H)-quinolinyl)ethoxy]phenyl}-2-ethoxypropanoate,
ethyl 4-[[1-(2-{4-[(2S)-2,3-diethoxy-3-oxopropyl]phenoxy}ethyl)-4,4-dimethyl-1,2,3,4-tetrahydro-6-quinolinyl](methoxyimino)methyl]benzoate,
4-[[1-(2-{4-[(2S)-2-carboxy-2-ethoxyethyl]phenoxy}ethyl)-4,4-dimethyl-1,2,3,4-tetrahydro-6-quinolinyl](methoxyimino)methyl]benzoic acid,
ethyl 2-ethoxy-3-{4-[2-(6-[(hydroxyimino)(phenyl)methyl]-4,4,7-trimethyl-3,4-dihydro-1(2H)-quinolinyl)ethoxy]phenyl}propanoate,
2-ethoxy-3-{4-[2-(6-[(hydroxyimino)(phenyl)methyl]-4,4,7-trimethyl-3,4-dihydro-1(2H)-quinolinyl)ethoxy]phenyl}propanoic acid,
ethyl 3-{4-[2-(6-[cyclohexyl(hydroxyimino)methyl]-4,4-dimethyl-3,4-dihydro-1(2H)-quinolinyl)ethoxy]phenyl}-2(S)-ethoxypropanoate,
3-{4-[2-(6-[cyclohexyl(hydroxyimino)methyl]-4,4-dimethyl-3,4-dihydro-1(2H)-quinolinyl)ethoxy]phenyl}-2(S)-ethoxypropanoic acid.

The enantiomers, diastereoisomers, and also pharmaceutically acceptable addition salts with an acid or a base of the preferred compounds of the invention form an integral part of the invention.

The present invention relates also to a process for the preparation of the compounds of formula (I) which is characterised in that there is used as starting material a compound of formula (III):

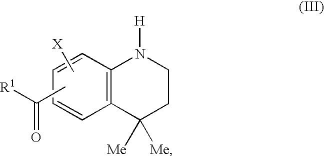

wherein R¹ and X are as defined for formula (I),
which is condensed in basic medium with a compound of formula (IV):

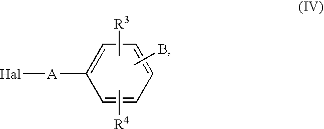

wherein A, B, R³ and R⁴ are as defined for formula (I) and Hal represents a halogen atom, to yield a compound of formula (V):

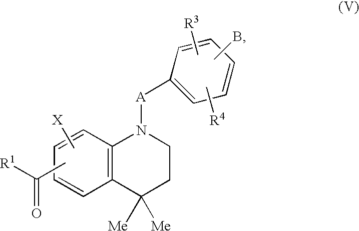

wherein R¹, R³, R⁴, X, A and B are as defined for formula (I),
which is subjected to the action of a compound of formula R²O—NH₂ wherein R² is as defined for formula (I) to yield a compound of formula (I):

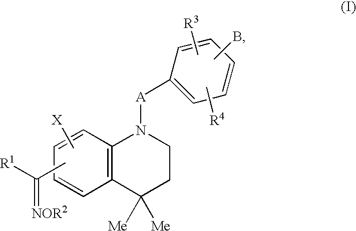

which may be purified according to a conventional separation technique, is converted, if desired, into addition salts with a pharmaceutically acceptable acid or base, and is optionally separated into isomers according to a conventional separation technique.

An advantageous variant relates to a process for the preparation of the compounds of formula (I) which is characterised in that there is used as starting material a compound of formula (III):

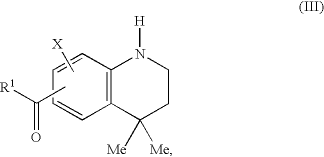

wherein R¹ and X are as defined for formula (I),
which is condensed with a compound of formula R²O—NH₂ wherein R² is as defined for formula (I) to yield a compound of formula (VI):

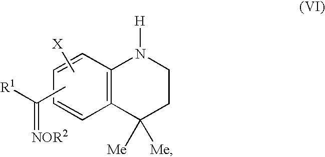

wherein R¹, R² and X are as defined for formula (I),
which is condensed in basic medium with a compound of formula (IV):

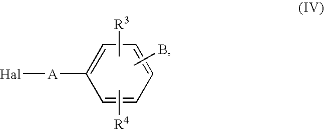

wherein A, B, R³ and R⁴ are as defined for formula (I) and Hal represents a halogen atom, to yield a compound of formula (I):

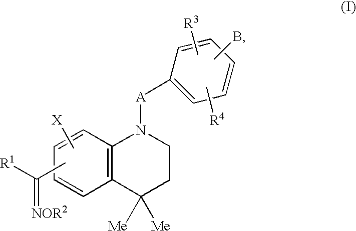

which may be purified according to a conventional separation technique, is converted, if desired, into addition salts with a pharmaceutically acceptable acid or base, and is optionally separated into isomers according to a conventional separation technique.

Another advantageous variant concerns a process for the preparation of the compounds of formula (I) wherein A represents an alkyleneoxy chain which is characterised in that there is used as starting material a compound of formula (VII)

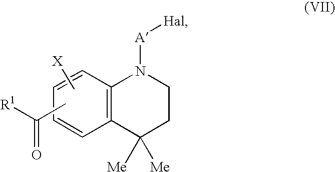

wherein R¹ and X are as defined for formula (I), A′ represents a (C₁-C₆)alkylene chain and Hal represents a halogen atom,
which is condensed in basic medium with a compound of formula (VIII):

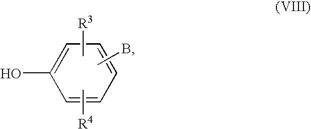

wherein B, R³ and R⁴ are as defined for formula (I), to yield a compound of formula (IX):

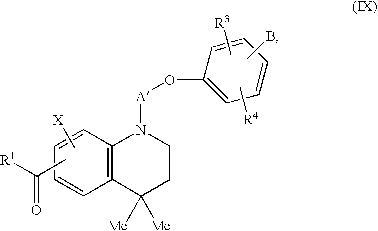

wherein R¹, X, B, R³ and R⁴ are as defined for formula (I) and A′ represents a (C₁-C₆)-alkylene chain,
which is condensed with a compound of formula R²O—NH₂ wherein R² is as defined for formula (I) to yield a compound of formula (I/a), a particular case of the compounds of formula (I):

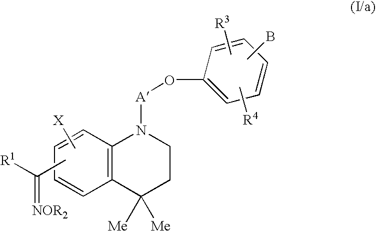

wherein R¹, R², X, B, R³ and R⁴ are as defined for formula (I) and A′ represents a (C₁-C₆)-alkylene chain,
which may be purified according to a conventional separation technique, is converted, if desired, into addition salts with a pharmaceutically acceptable acid or base, and is optionally separated into isomers according to a conventional separation technique.

The compounds of formula (III) or (VII) are commercial products, or readily obtainable by the person skilled in the art by conventional chemical reactions, or described in the literature.

The invention relates also to the compounds of formula (V)

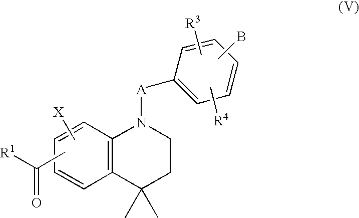

wherein R¹, R³, R⁴, X, A and B are as defined for the compounds of formula (I) for use as intermediates for the synthesis of the compounds of formula (I) and as hypoglycaemic and hypolipaemic agents.

The compounds of the present invention have very valuable pharmacological properties.

The compounds demonstrate especially an excellent activity in lowering blood glucose levels. As a result of such properties they can be used therapeutically in the treatment and/or prophylaxis of hyperglycaemia, dyslipidaemia and, more especially, in the treatment of non-insulin-dependent type II diabetes, glucose intolerance, disorders associated with syndrome X (including hypertension, obesity, insulin resistance, atherosclerosis, hyperlipidaemia), coronary artery disease and other cardiovascular diseases (including arterial hypertension, cardiac insufficiency, venous insufficiency), renal disorders (including glomerulonephritis, glomerulosclerosis, nephrotic syndrome, hypertensive nephrosclerosis), retinopathy, disorders associated with the activation of endothelial cells, psoriasis, polycystic ovary syndrome, dementia, diabetic complications and osteoporosis.

They can be used as aldose reductase inhibitors for improving cognitive functions in dementia and for the complications of diabetes, intestinal inflammatory disorders, myotonic dystrophy, pancreatitis, arteriosclerosis, xanthoma.

The activity of these compounds is also recommended for the treatment and/or prophylaxis of other diseases, including type I diabetes, hypertriglyceridaemia, syndrome X, insulin resistance, dyslipidaemia in diabetics, hyperlipidaemia, hypercholesterolaemia, arterial hypertension, cardiac insufficiency, and cardiovascular disease, especially atherosclerosis.

The compounds are furthermore indicated for use in the regulation of appetite, especially in the regulation of food intake in subjects suffering from disorders such as obesity, anorexia, bulimia and anorexia nervosa.

The compounds can accordingly be used in the prevention or treatment of hypercholesterolaemia, obesity with advantageous effects on hyperlipidaemia, hyperglycaemia, osteoporosis, glucose intolerance, insulin resistance or disorders in which insulin resistance is a secondary physiopathological mechanism.

The use of those compounds enables reduction of total cholesterol, body weight, leptin resistance, plasma glucose, triglycerides, LDLs, VLDLs and also plasma free fatty acids.

The compounds can be used in association with HMG CoA reductase inhibitors, fibrates, nicotinic acid, cholestyramine, colestipol, probucol, GLP1, metformin, biguanides or glucose reabsorption inhibitors and can be administered together or at different times to act in synergy in the patient treated.

They furthermore exhibit activity in cancer pathologies and especially hormone-dependent cancers, such as breast cancer and colon cancer, and also have an inhibiting effect on the angiogenesis processes implicated in those pathologies.

Amongst the pharmaceutical compositions according to the invention there may mentioned more especially those which are suitable for oral, parenteral, nasal, per- or trans-cutaneous, rectal, perlingual, ocular or respiratory administration and especially tablets or dragées, sublingual tablets, sachets, paquets, gelatin capsules, glossettes, lozenges, suppositories, creams, ointments, dermal gels and drinkable or injectable ampoules.

The dosage varies in accordance with the sex, age and weight of the patient, the administration route, the nature of the therapeutic indication or of any associated treatments and ranges from 0.1 mg to 1 g per 24 hours taken in 1 or more administrations.

The present invention relates also to a new association between a heterocyclic compound of formula (I) or of formula (V) as defined hereinbefore and an antioxidant agent for obtaining pharmaceutical compositions for use in the treatment and/or prevention of obesity and overweight characterised by a body mass index greater than 25. The antioxidant agents according to the invention are, more especially, anti-free radical agents or free-radical trapping agents, antilipoperoxidant agents, chelating agents or agents capable of regenerating endogenous antioxidants such as glutathione, vitamin C or vitamin E, and also addition salts thereof with a pharmaceutically acceptable acid or base.

The antioxidant agent of the association according to the invention is more preferably represented by quinone compounds such as ubiquinone or coenzyme Q₁₀, which acts as a free-radical trapping agent but which is also capable of regenerating vitamin E.

The following are preferred associations according to the invention:
(2S)-2-ethoxy-3-{4-[2-(6-[(methoxyimino)(phenyl)methyl]-4,4-dimethyl-3,4-dihydro-1(2H)-quinolinyl)ethoxy]phenyl}propanoic acid and coenzyme Q₁₀,
3-{4-[2-(6-(cyclohexylcarbonyl)-4,4-dimethyl-3,4-dihydro-1(2H)-quinolinyl)ethoxy]-phenyl}-2-ethoxypropanoic acid and coenzyme Q₁₀.

Furthermore, the association according to the invention has entirely surprising pharmacological properties: the Applicant has, in fact, demonstrated that a synergy exists between the two compounds of the association allowing a very significant reduction in body fat to be obtained, making it useful in the treatment and/or prevention of obesity and of overweight characterised by a body mass index greater than 25.

In the United States, obesity affects 20% of men and 25% of women. Patients having a body mass index (BMI=weight (kg)/height² (m²)) greater than or equal to 30 are considered to be obese (Int. J. Obes., 1998, 22, 39-47; Obesity Lancet, 1997, 350, 423-426). Obesity (BMI≧30) and overweight (25<BMI<30) can have various origins: they may come about following deregulation of food intake, following hormonal disturbance, or following administration of a treatment: treating type II diabetes with sulphonylureas causes patients to gain weight. Similarly, in type I (insulin-dependent) diabetes, insulin therapy is also a cause of weight gain in patients (In Progress in Obesity Research, 8^{th} International Congress on Obesity, 1999, 739-746; Annals of Internal Medicine, 1998, 128, 165-175).

Obesity and overweight are well-established risk factors for cardiovascular diseases: they are associated with a significant increase in the risk of cerebro-vascular accidents and non-insulin-dependent diabetes, because they predispose to insulin resistance, to dyslipidaemia and to the appearance of macrovascular disorders (nephropathy, retinopathy, angiopathy). Further pathologies are the consequence of obesity or overweight: there may be mentioned, in particular, vesicular calculi, respiratory dysfunction, several forms of cancer and, in the case of very severe obesity, premature death (N. Engl. J. Med., 1995, 333, 677-385; JAMA, 1993, 270, 2207-2212).

The association according to the invention allows a weight loss to be obtained which, even if moderate, significantly reduces all the risk factors associated with obesity (Int. J. Obes., 1997, 21, 55-9; Int. J. Obes., 1992, 21, S5-9).

The association according to the invention will therefore be found to be useful in the treatment and/or prevention of obesity and of overweight characterised by a body mass index greater than 25.

The invention accordingly relates to the use of the association between a compound of formula (I) or formula (V) and an antioxidant agent in obtaining pharmaceutical compositions intended for the treatment and/or prevention of obesity and of overweight characterised by a body mass index greater than 25 and less than 30.

In particular, the association according to the invention is useful in the treatment and/or prevention of obesity and of overweight characterised by a body mass index greater than 25 and less than 30 caused by a therapeutic treatment, such as treatment for type I or type II diabetes.

The invention accordingly relates to the use of the association between a compound of formula (I) or of formula (V) and an antioxidant agent in obtaining pharmaceutical compositions intended for the treatment and/or prevention of obesity and of overweight characterised by a body mass index greater than 25 and less than 30 caused by a therapeutic treatment, such as treatment for type I or type II diabetes.

The invention relates also to pharmaceutical compositions comprising the association between a compound of formula (I) or of formula (V) and an antioxidant agent, as defined hereinbefore, in combination with one or more pharmaceutically acceptable excipients.

Among the pharmaceutical compositions according to the invention there may be mentioned, more especially, those that are suitable for oral, parenteral or nasal administration, tablets or dragées, sublingual tablets, gelatin capsules, lozenges, suppositories, creams, ointments, dermal gels, etc.

In particular, the invention relates to pharmaceutical compositions comprising a compound of formula (I) or of formula (V) as defined hereinbefore and an antioxidant agent, such as coenzyme Q₁₀ or vitamin E, in combination with one or more pharmaceutically acceptable excipients.

The dosage used varies according to the sex, age and weight of the patient, the administration route, the nature of the therapeutic indication or of any associated treatments and ranges from 0.1 mg to 1 g of each component of the association per 24 hours in one or more administrations.

The Preparations and Examples which follow illustrate the invention but do not limit it in any way.

### Preparation 1: Ethyl 3-{4-[2-(6-benzoyl-4,4-dimethyl-1,2,3,4-tetrahydro-1-quinolinyl)ethoxy]phenyl}-2-ethoxypropanoate

### Step A: N-(2-Bromoethyl)-4,4-dimethyl-1,2,3,4-tetrahydroquinoline

Under an argon atmosphere, 5.1 ml of bromoacetyl bromide are added dropwise to a solution of 6.3 g of 4,4-dimethyl-1,2,3,4-tetrahydroquinoline in anhydrous THF at 0° C. 8.2 ml of triethylamine are then added dropwise, bringing about the formation of a precipitate. The mixture is maintained under vigorous stirring for one hour before being hydrolysed and extracted with ethyl acetate. The organic phase is washed in succession with water and with a saturated sodium chloride solution, dried over magnesium sulphate, filtered and concentrated under reduced pressure. Under an argon atmosphere, the residual oils are dissolved in anhydrous toluene and cooled to 0° C. 98 ml of a solution of borohydride complexed with 1M THF in THF are slowly added. After returning to ambient temperature, the solution is stirred for 2 hours and then hydrolysed with a saturated sodium hydrogen carbonate solution. After being stirred for 15 minutes, the mixture is extracted with ethyl acetate. The organic phase is washed with water and with saturated sodium chloride solution, dried over magnesium sulphate and filtered. The solvents are evaporated off under reduced pressure and the crude reaction product is purified by chromatography on silica gel to yield the title compound in the form of a colourless oil.

MS (EI): m/z=267 (M⁷⁹Br); 268 (M⁸⁰Br); 174.

### Step B: N-(2-Bromoethyl)-4,4-dimethyl-6-benzoyl-1,2,3,4-tetrahydroquinoline

Under an inert atmosphere, 1.8 ml of titanium tetrachloride are added dropwise to a solution of 1.5 g of the compound obtained in Step A and 1.9 ml of benzoyl chloride in 7 ml of anhydrous 1,2-DCE. The mixture is heated at reflux for 18 hours. After returning to ambient temperature, the mixture is hydrolysed over ice-water and extracted twice with dichloromethane. The combined organic phases are washed with a saturated sodium hydrogen carbonate solution, dried over magnesium sulphate, filtered and concentrated under reduced pressure. The title product is obtained in the form of a yellow oil after purification by chromatography on silica gel (PE/AcOEt:9/1 then 8/2).

MS (SI): m/z=371 (M+H⁷⁹Br); 372 (M+H⁸⁰Br).

### Step C: Ethyl 3-{4-[2-(6-benzoyl-4,4-dimethyl-1,2,3,4-tetrahydro-1-quinolinyl)-ethoxy]phenyl}-2-ethoxypropanoate

Under an inert atmosphere, 2.15 g of potassium carbonate are added in one portion to a solution of 371 mg of ethyl 2-ethoxy-3-(4-hydroxyphenyl)propanoate in anhydrous DMF. The suspension is stirred for 30 minutes and then 870 mg of the compound obtained in Step B in solution in anhydrous DMF are added. The mixture is heated at 80° C. for from 24 to 48 hours. Following evaporation of most of the DMF, the residue is taken up in ethyl acetate and hydrolysed. The organic phase is washed with an aqueous 10% sodium hydroxide solution, dried over magnesium sulphate, filtered and concentrated under reduced pressure. The crude reaction product is then purified by chromatography on silica gel (PE/AcOEt:9/1 then 8/2) to yield the title product in the form of a yellow oil.

MS (EI): m/z=529 (M); 307; 173.

### Preparation 2: Ethyl 3-{4-[2-(6-benzoyl-4,4-dimethyl-1,2,3,4-tetrahydro-1-quinolinyl)ethoxy]phenyl}-2(S)-ethoxypropanoate

Under an inert atmosphere, 5.69 g of potassium carbonate are added in one portion to a solution of 981 mg of ethyl 2(S)-ethoxy-3-(4-hydroxyphenyl)propanoate in anhydrous DMF. The suspension is stirred for 30 minutes and then 2.3 g of the compound obtained in Step B of Preparation 1 in solution in anhydrous DMF are added. The mixture is heated at 80° C. for from 24 to 48 hours. Following evaporation of most of the DMF, the residue is taken up in ethyl acetate and hydrolysed. The organic phase is washed with an aqueous 10% sodium hydroxide solution, dried over magnesium sulphate, filtered and concentrated under reduced pressure. The crude reaction product is then purified by chromatography on silica gel (PE/AcOEt:9/1 then 8/2) to yield the title product in the form of a yellow oil.

MS (EI): m/z 529 (M); 307; 173.

### Preparation 3: Ethyl 3-{4-[2-(6-benzoyl-4,4-dimethyl-3,4-dihydro(2H)-1-quinolinyl)-ethoxy]phenyl}-2-(4-trifluoromethylphenoxy)propanoate

Under an inert atmosphere, 2.19 g of potassium carbonate are added in one portion to a solution of 560 mg of ethyl 3-(4-hydroxyphenyl)-2-(4-trifluoromethylphenoxy)propanoate in anhydrous DMF. The suspension is stirred for 30 minutes and then 883 mg of the compound obtained in Step B of Preparation 1 in solution in anhydrous DMF are added. The mixture is heated at 80° C. for from 24 to 48 hours. Following evaporation of most of the DMF, the residue is taken up in ethyl acetate and hydrolysed. The organic phase is washed with an aqueous 10% sodium hydroxide solution, dried over magnesium sulphate, filtered and concentrated under reduced pressure. The crude reaction product is then purified by chromatography on silica gel (PE/AcOEt:9/1 then 8/2) to yield the title product in the form of a yellow oil.

MS (EI): m/z=645 (M); 278; 105.

### Preparation 4: 2(S)-Acetoxy-3-{4-[2-(6-benzoyl-4,4-dimethyl-3,4-dihydro(2H)-1-quinolinyl)ethoxy]phenyl}propanoic acid

Under an inert atmosphere, 2.62 g of potassium carbonate are added in one portion to a solution of 425 mg of 2(S)-acetoxy-3-(4-hydroxyphenyl)propanoic acid in anhydrous DMF. The suspension is stirred for 30 minutes and then 1.06 g of the compound obtained in Step B of Preparation 1 in solution in anhydrous DMF are added. The mixture is heated at 80° C. for from 24 to 48 hours. Following evaporation of most of the DMF, the residue is taken up in ethyl acetate and hydrolysed. The organic phase is washed with an aqueous 10% sodium hydroxide solution, dried over magnesium sulphate, filtered and concentrated under reduced pressure. The crude reaction product is then purified by chromatography on silica gel (PE/AcOEt:8/2) to yield the title product in the form of a yellow mousse.

Melting point: 60-62° C.

### Preparation 5: Ethyl 3-{4-[2-(6-benzoyl-4,4-dimethyl-3,4-dihydro(2H)-1-quinolinyl)-ethoxy]phenyl}-2-(2,2,2-trifluoroethoxy)propanoate

Under an inert atmosphere, 1.79 g of potassium carbonate are added in one portion to a solution of 380 mg of ethyl 3-(4-hydroxyphenyl)-2-(2,2,2-trifluoroethoxy)propanoate in anhydrous DMF. The suspension is stirred for 30 minutes and then 725 mg of the compound obtained in Step B of Preparation 1 in solution in anhydrous DMF are added. The mixture is heated at 80° C. for from 24 to 48 hours. Following evaporation of most of the DMF, the residue is taken up in ethyl acetate and hydrolysed. The organic phase is washed with an aqueous 10% sodium hydroxide solution, dried over magnesium sulphate, filtered and concentrated under reduced pressure. The crude reaction product is then purified by chromatography on silica gel (PE/AcOEt:9/1 then 8/2) to yield the title product in the form of a yellow mousse.

Melting point: 56-58° C.

### Preparation 6: Ethyl 3-{4-[2-(6-benzoyl-4,4-dimethyl-3,4-dihydro(2H)-1-quinolinyl)-ethoxy]phenyl}-2-(2-benzoylphenylamino)propanoate

Under an inert atmosphere, 1.23 g of potassium carbonate are added in one portion to a solution of 697 mg of ethyl 2-(2-benzoylphenylamino)-3-(4-hydroxyphenyl)propanoate in anhydrous DMF. The suspension is stirred for 30 minutes and then 1 g of the compound obtained in Step B of Preparation 1 in solution in anhydrous DMF are added. The mixture is heated at 80° C. for from 24 to 48 hours. Following evaporation of most of the DMF, the residue is taken up in ethyl acetate and hydrolysed. The organic phase is washed with an aqueous 10% sodium hydroxide solution, dried over magnesium sulphate, filtered and concentrated under reduced pressure. The crude reaction product is then purified by chromatography on silica gel (PE/AcOEt:95/5 then 75/25) to yield the title product in the form of a yellow mousse.

Melting point: 75-77° C.

### Preparation 7: Ethyl 3-(4-{2-[6-(4-cyanobenzoyl)-4,4-dimethyl-3,4-dihydro(2H)-1-quinolinyl]ethoxy}phenyl)-2(S)-ethoxypropanoate

Step A: 4-[1-(2-Bromoethyl)-4,4-dimethyl-1,2,3,4-tetrahydroquinoline-6-carbonyl]benzonitrile

Under an inert atmosphere, 0.74 ml of titanium tetrachloride are added dropwise to a solution of 900 mg of the compound obtained in Step A of Preparation 1 and 1.11 g of 4-cyanobenzoyl chloride in 5 ml of anhydrous 1,2-DCE. The mixture is heated at reflux for 18 hours. After returning to ambient temperature, the mixture is hydrolysed over ice-water and extracted twice with dichloromethane. The combined organic phases are washed with a saturated sodium hydrogen carbonate solution, dried over magnesium sulphate, filtered and concentrated under reduced pressure. The title product is obtained in the form of a brown oil after purification by chromatography on silica gel (PE/AcOEt:9/1 then 8/2).

MS (EI): m/z=397 (M); 303.

### Step B: Ethyl 3-(4-[2-[6-(4-cyanobenzoyl)-4,4-dimethyl-3,4-dihydro(2H)-1-quinolinyl]ethoxy]phenyl)-2(S)-ethoxypropanoate

Under an inert atmosphere, 2.15 g of potassium carbonate are added in one portion to a solution of 371 mg of ethyl 2(S)-ethoxy-3-(4-hydroxyphenyl)propanoate in anhydrous DMF. The suspension is stirred for 30 minutes and then 870 mg of the compound obtained in Step A in solution in anhydrous DMF are added. The mixture is heated at 80° C. for from 24 to 48 hours. Following evaporation of most of the DMF, the residue is taken up in ethyl acetate and hydrolysed. The organic phase is washed with an aqueous 10% sodium hydroxide solution, dried over magnesium sulphate, filtered and concentrated under reduced pressure. The crude reaction product is then purified by chromatography on silica gel (PE/AcOEt:95/5 then 75/25) to yield the title product in the form of a yellow oil.

MS (EI): m/z=554 (M); 303; 130.

### Preparation 8: Ethyl 4-(1-{2-[4-(2(S)-ethoxy-2-ethoxycarbonylethyl)phenoxy]ethyl}-4,4-dimethyl-1,2,3,4-tetrahydroquinoline-6-carbonyl)benzoate

### Step A: Ethyl 4-[1-(2-bromoethyl)-4,4-dimethyl-1,2,3,4-tetrahydroquinoline-6-carbonyl]benzoate

Under argon, 2.5 g of the compound obtained in Step A of Preparation 1 are dissolved in 15 ml of anhydrous 1,2-DCE and then 4.73 g of terephthaloyl chloride and 2.56 ml of titanium tetrachloride are added dropwise in succession. The resulting solution is heated at reflux for 18 hours. After returning to ambient temperature, 20 ml of absolute ethanol are added and the mixture is again heated at reflux for 1 hour. After returning to ambient temperature, the mixture is hydrolysed over ice-water and extracted twice with dichloromethane. The combined organic phases are washed with a saturated sodium hydrogen carbonate solution, dried over magnesium sulphate, filtered and concentrated under reduced pressure. The title compound is obtained in the form of a green oil after chromatography on silica gel (PE/AcOEt:95/5 then 8/2).

MS (EI): m/z=443 (M⁷⁹Br); 444 (M⁸⁰Br); 350; 177; 149.

### Step B: Ethyl 4-(1-{2-[4-(2(S)-ethoxy-2-ethoxycarbonylethyl)phenoxy]ethyl}-4,4-dimethyl-1,2,3,4-tetrahydroquinoline-6-carbonyl)benzoate

Under an inert atmosphere, 5.24 g of potassium carbonate are added in one portion to a solution of 902 mg of ethyl 2(S)-ethoxy-3-(4-hydroxyphenyl)propanoate in anhydrous DMF. The suspension is stirred for 30 minutes and then 2.5 g of the compound obtained in Step A in solution in anhydrous DMF are added. The mixture is heated at 80° C. for from 24 to 48 hours. Following evaporation of most of the DMF, the residue is taken up in ethyl acetate and hydrolysed. The organic phase is washed with an aqueous 10% sodium hydroxide solution, dried over magnesium sulphate, filtered and concentrated under reduced pressure. The crude reaction product is then purified by chromatography on silica gel (PE/AcOEt:95/5) to yield the title product in the form of a green oil.

MS (EI): m/z=350; 177; 149; 107; 43.

### Preparation 9: Ethyl 3-{(4-[2-(6-benzoyl-4,4,7-trimethyl-3,4-dihydro-1(2H)-quinolinyl)ethoxy]phenyl}-2(S)-ethoxypropanoate

### Step A: 1-(2-Bromoethyl)-4,4,7-trimethyl-1,2,3,4-tetrahydroquinoline

The procedure is as in Step A of Preparation 1 with the replacement of 4,4-dimethyl-1,2,3,4-tetrahydroquinoline with 4,4,7-trimethyl-1,2,3,4-tetrahydroquinoline. Colourless oil.

MS (EI): m/z=160; 281 (M⁷⁹Br); 283 (M⁸¹Br).

### Step B: N-(2-Bromoethyl)-4,4,7-trimethyl-6-benzoyl-1,2,3,4-tetrahydroquinoline

The procedure is as in Step B of Preparation 1 starting from the compound obtained in Step A.

### Orangey oil.

MS (EI):m/z=44; 77; 105; 292; 341; 385(M⁷⁹Br); 387(M⁸¹Br).

### Step C: Ethyl 3-{4-[2-(6-benzoyl-4,4,7-trimethyl-3,4-dihydro-1(2H)-quinolinyl)-ethoxy]phenyl}-2(S)-ethoxypropanoate

The procedure is as in Preparation 2 starting from the compound obtained in Step B.

### Yellow oil.

MS (EI): m/z=544 (M+H); 561 (M+NH₄); 566 (M+Na).

### Preparation 10: 3-{4-[2-(6-Benzoyl-4,4,7-trimethyl-3,4-dihydro-1(2H)-quinolinyl)ethoxy]phenyl}-2(S)-ethoxypropanoic Acid

The procedure is as in Preparation 2 starting from the compound obtained in Preparation 9 and with the replacement of ethyl 2(S)-ethoxy-3-(4-hydroxyphenyl)propanoate with 2(S)-ethoxy-3-(4-hydroxyphenyl)propanoic acid.

### Preparation 11: Ethyl 3-{4-[2-(6-(cyclohexylcarbonyl)-4,4-dimethyl-3,4-dihydro-1(2H)-quinolinyl)ethoxy]phenyl}-2-ethoxypropanoate

The procedure is as in Preparation 1 with the replacement of benzoyl chloride with cyclohexylcarbonyl chloride in Step B.

MS (EI): m/z=536 (M+H).

### Preparation 12: 3-{4-[2-(6-(Cyclohexylcarbonyl)-4,4-dimethyl-3,4-dihydro-1(2H)-quinolinyl)ethoxy]phenyl}-2-ethoxypropanoic acid

The procedure is as in Preparation 2 starting from the compound obtained in Preparation 11.

MS (EI): m/z=508 (M+H); 530 (M+Na).

### EXAMPLE 1

### Ethyl 2-ethoxy-3-{4-[2-(6-[(hydroxyimino)(phenyl)methyl]-4,4-dimethyl-3,4-dihydro-1(2H)-quinolinyl)ethoxy]phenyl}propanoate

Under an inert atmosphere, 350 mg of the compound obtained in Preparation 1 are dissolved in 5 ml of pyridine and then 460 mg of hydroxylamine hydrochloride are added in one portion. The solution is heated at reflux for 3 hours. After returning to ambient temperature, the solvents are evaporated off under reduced pressure. The residue is taken up in ethyl acetate and washed with water and then with a saturated ammonium chloride solution. The organic phase is dried over magnesium sulphate, filtered and concentrated under reduced pressure. The crude reaction product is purified by chromatography on silica gel (heptane/AcOEt:9/1) to yield the title product in the form of a yellow mousse.

Melting point: 46-48° C.

Elemental microanalysis:

| | C | H | N |
|---|---|---|---|
| % theoretical | 72.77 | 7.40 | 5.14 |
| % experimental | 72.69 | 7.29 | 5.11 |

### EXAMPLE 2

### Ethyl 2-ethoxy-3-{4-[2-(6-[(methoxyimino)(phenyl)methyl]-4,4-dimethyl-3,4-dihydro-1(2H)-quinolinyl)ethoxy]phenyl}propanoate

Under an inert atmosphere, 550 mg of the compound obtained in Preparation 1 are dissolved in 10 ml of pyridine and then 868 mg of methoxylamine hydrochloride are added in one portion. The solution is heated at reflux for 2 hours. After returning to ambient temperature, the solvents are evaporated off under reduced pressure. The residue is taken up in ethyl acetate and washed with water and then with a saturated ammonium chloride solution. The organic phase is dried over magnesium sulphate, filtered and concentrated under reduced pressure. The crude reaction product is purified by chromatography on silica gel (heptane/AcOEt:9/1) to yield the title product in the form of an orangey-yellow oil.

Elemental microanalysis.

| | C | H | N |
|---|---|---|---|
| % the oretical | 73.09 | 7.58 | 5.01 |
| % experimental | 73.19 | 7.80 | 5.14 |

### EXAMPLE 3

### 2-Ethoxy-3-{4-[2-(6-[(methoxyimino)(phenyl)methyl]-4,4-dimethyl-3,4-dihydro-1(2H)-quinolinyl)ethoxy]phenyl}propanoic acid

200 mg of the compound obtained in Example 2 are dissolved in 5 ml of THF and the resulting solution is cooled to 0° C. before the addition, in 2 ml portions, of 20 ml of an aqueous 0.1N lithium hydroxide solution. After returning to ambient temperature, the mixture is stirred for 24 hours and then the pH of the solution is adjusted to 2 by addition of an aqueous 1N hydrochloric acid solution. After extractions with dichloromethane, the combined organic phases are dried over magnesium sulphate and filtered, and the solvents are evaporated off under reduced pressure. The residue is purified by chromatography on silica gel (heptane/AcOEt:8/2+1% AcOH) to yield the title product in the form of a brown mousse.

Melting point: 62-64° C.

Elemental microanalysis:

| | C | H | N |
|---|---|---|---|
| % theoretical | 72.43 | 7.22 | 5.28 |
| % experimental | 72.17 | 7.07 | 5.21 |

### EXAMPLE 4

### Ethyl (2S)-2-ethoxy-3-{4-[2-(6-[(methoxyimino)(phenyl)methyl]-4,4-dimethyl-3,4-dihydro-1(2H)-quinolinyl)ethoxy]phenyl}propanoate

Under an inert atmosphere, 350 mg of the compound obtained in Preparation 2 are dissolved in 8 ml of pyridine and then 552 mg of methoxylamine hydrochloride are added in one portion. The solution is heated at reflux for 2 hours. After returning to ambient temperature, the solvents are evaporated off under reduced pressure. The residue is taken up in ethyl acetate and washed with water and then with a saturated ammonium chloride solution. The organic phase is dried over magnesium sulphate, filtered and concentrated under reduced pressure. The crude reaction product is purified by chromatography on silica gel (heptane/AcOEt:9/1) to yield the title product in the form of a orangey-yellow oil.

MS (SI): m/z=559 (M+H); 581 (M+Na)

Elemental microanalysis.

| | C | H | N |
|---|---|---|---|
| % theoretical | 73.09 | 7.58 | 5.01 |
| % experimental | 72.98 | 7.66 | 5.03 |

### EXAMPLE 5

### (2S)-2-Ethoxy-3-{4-[2-(6-[(methoxyimino)(phenyl)methyl]-4,4-dimethyl-3,4-dihydro-1(2H)-quinolinyl)ethoxy]phenyl}propanoic acid

270 mg of the compound obtained in Example 4 are dissolved in 7 ml of THF and the resulting solution is cooled to 0° C. before the addition, in 2 ml portions, of 26 ml of an aqueous 0.1N lithium hydroxide solution. After returning to ambient temperature, the mixture is stirred for 24 hours and then the pH of the solution is adjusted to 2 by addition of an aqueous 1N hydrochloric acid solution. After extractions with dichloromethane, the combined organic phases are dried over magnesium sulphate and filtered, and the solvents are evaporated off under reduced pressure. The residue is purified by chromatography on silica gel (heptane/AcOEt:8/2+1% AcOH) to yield the title product in the form of a brown mouse.

Melting point: 62-64° C.

Elemental microanalysis:

| | C | H | N |
|---|---|---|---|
| % theoretical | 72.43 | 7.22 | 5.28 |
| % experimental | 72.59 | 7.21 | 5.17 |

### EXAMPLE 6

### Ethyl 3-{4-[2-(6-[(methoxyimino)(phenyl)methyl]-4,4-dimethyl-3,4-dihydro-1(2H)-quinolinyl)ethoxy]phenyl}-2-[4-(trifluoromethyl)-phenoxy]propanoate

Under an inert atmosphere, 300 mg of the compound obtained in Preparation 3 are dissolved in 7 ml of pyridine and then 388 mg of methoxylamine hydrochloride are added in one portion. The solution is heated at reflux for 3 hours. After returning to ambient temperature, the solvents are evaporated off under reduced pressure. The residue is taken up in ethyl acetate and washed with water and then with a saturated ammonium chloride solution. The organic phase is dried over magnesium sulphate, filtered and concentrated under reduced pressure. The crude reaction product is purified by chromatography on silica gel (heptane/AcOEt:9/1) to yield the title product in the form of a white mousse.

Melting point: 44-46° C.

Elemental microanalysis:

| | C | H | N |
|---|---|---|---|
| % theoretical | 69.42 | 6.12 | 4.15 |
| % experimental | 69.81 | 6.23 | 4.32 |

### EXAMPLE 7

### 2-(Acetoxy)-3-{4-[2-(6-[(methoxyimino)(phenyl)methyl]-4,4-dimethyl-3,4-dihydro-1(2H)-quinolinyl)ethoxy]phenyl}propanoic acid

Under an inert atmosphere, 530 mg of the compound obtained in Preparation 4 are dissolved in 8 ml of pyridine and then 858 mg of methoxylamine hydrochloride are added in one portion. The solution is heated at reflux for 5 hours. After returning to ambient temperature, the solvents are evaporated off under reduced pressure. The residue is taken up in ethyl acetate and washed with water and then with a saturated ammonium chloride solution. The organic phase is dried over magnesium sulphate, filtered and concentrated under reduced pressure. The crude reaction product is purified by chromatography on silica gel (heptane/AcOEt:8/2) to yield the title product in the form of a pale yellow mousse.

Melting point: 64-66° C.

Elemental microanalysis:

| | C | H | N |
|---|---|---|---|
| % theoretical | 70.57 | 6.66 | 5.14 |
| % experimental | 70.24 | 6.80 | 5.14 |

### EXAMPLE 8

### Ethyl 3-{4-[2-(6-[(methoxyimino)(phenyl)methyl]-4,4-dimethyl-3,4-dihydro-1(2H)-quinolinyl)ethoxy]phenyl}-2-(2,2,2-trifluoroethoxy)-propanoate

Under an inert atmosphere, 500 mg of the compound obtained in Preparation 5 are dissolved in 10 ml of pyridine and then 858 mg of methoxylamine hydrochloride are added in one portion. The solution is heated at reflux for 2 hours. After returning to ambient temperature, the solvents are evaporated off under reduced pressure. The residue is taken up in ethyl acetate and washed with water and then with a saturated ammonium chloride solution. The organic phase is dried over magnesium sulphate, filtered and concentrated under reduced pressure. The crude reaction product is purified by chromatography on silica gel (heptane/AcOEt:9/1) to yield the title product in the form of a yellow mousse.

Melting point: 58-60° C.

### EXAMPLE 9

### 3-{4-[2-(6-[(Methoxyimino)(phenyl)methyl]-4,4-dimethyl-3,4-dihydro-1(2H)-quinolinyl)ethoxy]phenyl}-2-(2,2,2-trifluoroethoxy)propanoic acid

400 mg of the compound obtained in Example 8 are dissolved in 10 ml of THF and the resulting solution is cooled to 0° C. before the addition, in 2 ml portions, of 36 ml of an aqueous 0.1N lithium hydroxide solution. After returning to ambient temperature, the mixture is stirred for 24 hours and then the pH of the solution is adjusted to 2 by addition of an aqueous 1N hydrochloric acid solution. After extractions with dichloromethane, the combined organic phases are dried over magnesium sulphate and filtered, and the solvents are evaporated off under reduced pressure. The residue is purified by chromatography on silica gel (heptane/AcOEt:8/2+1% AcOH) to yield the title product in the form of a brown mouse.

Melting point: 62-64° C.

Elemental microanalysis:

| | C | H | N |
|---|---|---|---|
| % theoretical | 65.74 | 6.03 | 4.79 |
| % experimental | 65.99 | 6.33 | 4.74 |

### EXAMPLE 10

### Ethyl 2-(2-benzoylanilino)-3-{4-[2-(6-[(methoxyimino)(phenyl)methyl]-4,4-dimethyl-3,4-dihydro-1(2H)-quinolinyl)ethoxy]phenyl}propanoate

Under an inert atmosphere, 540 mg of the compound obtained in Preparation 6 are dissolved in 10 ml of pyridine and then 663 mg of methoxylamine hydrochloride are added in one portion. The solution is heated at reflux for 2 hours. After returning to ambient temperature, the solvents are evaporated off under reduced pressure. The residue is taken up in ethyl acetate and washed with water and then with a saturated ammonium chloride solution. The organic phase is dried over magnesium sulphate, filtered and concentrated under reduced pressure. The crude reaction product is purified by chromatography on silica gel (heptane/AcOEt:9/1) to yield the title product in the form of a yellow mousse.

Melting point: 78-80° C.

### EXAMPLE 11

### 2-(2-Benzoylanilino)-3-{4-[2-(6-[(methoxyimino)(phenyl)methyl]-4,4-dimethyl-3,4-dihydro-1(2H)-quinolinyl)ethoxy]phenyl}propanoic acid

198 mg of the compound obtained in Example 10 are dissolved in 3 ml of THF and the resulting solution is cooled to 0° C. before the addition, in 2 ml portions, of 16 ml of an aqueous 0.1N lithium hydroxide solution. After returning to ambient temperature, the mixture is stirred for 24 hours and then the pH of the solution is adjusted to 2 by addition of an aqueous 1N hydrochloric acid solution. After extractions with dichloromethane, the combined organic phases are dried over magnesium sulphate and filtered, and the solvents are evaporated off under reduced pressure. The residue is purified by chromatography on silica gel (heptane/AcOEt:8/2+1% AcOH) to yield the title product in the form of an orange mousse.

Melting point: 100-102° C.

Elemental microanalysis:

| | C | H | N |
|---|---|---|---|
| % theoretical | 75.75 | 6.36 | 6.16 |
| % experimental | 75.51 | 6.59 | 5.92 |

### EXAMPLE 12

### Ethyl (2S)-3-{4-[2-(6-[(4-cyanophenyl)(methoxyimino)methyl]-4,4-dimethyl-3,4-dihydro-1(2H)-quinolinyl)ethoxy]phenyl}-2-ethoxypropanoate

Under an inert atmosphere, 175 mg of the compound obtained in Preparation 7 are dissolved in 6 ml of pyridine and then 263 mg of methoxylamine hydrochloride are added in one portion. The solution is heated at reflux for 5 hours. After returning to ambient temperature, the solvents are evaporated off under reduced pressure. The residue is taken up in ethyl acetate and washed with water and then with a saturated ammonium chloride solution. The organic phase is dried over magnesium sulphate, filtered and concentrated under reduced pressure. The crude reaction product is purified by chromatography on silica gel (heptane/AcOEt:95/5) to yield the title product in the form of a yellow oil.

MS (SI): m/z=584 (M+H); 601 (M+NH₄); 606 (M+Na).

### EXAMPLE 13

### Ethyl 4-[[1-(2-{4-[(2S)-2,3-diethoxy-3-oxopropyl]phenoxy}ethyl)-4,4-dimethyl-1,2,3,4-tetrahydro-6-quinolinyl](methoxyimino)methyl]-benzoate

Under an inert atmosphere, 1.15 g of the compound obtained in Preparation 8 are dissolved in 15 ml of pyridine and then 1.60 g of methoxylamine hydrochloride are added in one portion. The solution is heated at reflux for 6 hours. After returning to ambient temperature, the solvents are evaporated off under reduced pressure. The residue is taken up in ethyl acetate and washed with water and then with a saturated ammonium chloride solution. The organic phase is dried over magnesium sulphate, filtered and concentrated under reduced pressure. The crude reaction product is purified by chromatography on silica gel (heptane/AcOEt:85/15) to yield the title product in the form of an orangey-yellow oil.

MS (SI): m/z=631 (M+H).

### EXAMPLE 14

### 4-[[1-(2-{4-[(2S)-2-Carboxy-2-ethoxyethyl]phenoxy}ethyl)-4,4-dimethyl-1,2,3,4-tetrahydro-6-quinolinyl](methoxyimino)methyl]benzoic acid

600 mg of the compound obtained in Example 13 are dissolved in 6 ml of THF and the resulting solution is cooled to 0° C. before the addition, in 2 ml portions, of 95 ml of an aqueous 0.1N lithium hydroxide solution. After returning to ambient temperature, the mixture is stirred for 24 hours and then the pH of the solution is adjusted to 2 by addition of an aqueous 1N hydrochloric acid solution. After extractions with dichloromethane, the combined organic phases are dried over magnesium sulphate and filtered, and the solvents are evaporated off under reduced pressure. The residue is purified by chromatography on silica gel (DCM/MeOH:99/1) to yield the title product in the form of a pink mousse.

Melting point: 83-85° C.

### EXAMPLE 15

### Ethyl 2-ethoxy-3-{(4-[2-(6-[(hydroxyimino)(phenyl)methyl]-4,4,7-trimethyl-3,4-dihydro-1(2H)-quinolinyl)ethoxy]phenyl}propanoate

The procedure is as in Example 1 starting from the compound obtained in Preparation 9. Yellow oil.

MS (SI): m/z=559 (M+H); 581 (M+Na).

### EXAMPLE 16

### 2-Ethoxy-3-{4-[2-(6-[(hydroxyimino)(phenyl)methyl]-4,4,7-trimethyl-3,4-dihydro-1(2H)-quinolinyl)ethoxy]phenyl}propanoic acid

The procedure is as in Example 3 starting from the compound obtained in Example 15. Yellow mousse.

Melting point: 84-86° C.

### EXAMPLE 17

### Ethyl 3-{4-[2-(6-[cyclohexyl(hydroxyimino)methyl]-4,4-dimethyl-3,4-dihydro-1(2H)-quinolinyl)ethoxy]phenyl}-2(S)-ethoxypropanoate

The procedure is as in Example 1 starting from the compound obtained in Preparation 11.

### EXAMPLE 18

### 3-{4-[2-(6-[Cyclohexyl(hydroxyimino)methyl]-4,4-dimethyl-3,4-dihydro-1(2H)-quinolinyl)ethoxy]phenyl}-2(S)-ethoxypropanoic acid

The procedure is as in Example 3 starting from the compound obtained in Example 17.

### PHARMACOLOGICAL STUDY

### Example A

### Acute Toxicity Study

Acute toxicity was evaluated after oral administration to groups each comprising 8 mice (26±2 grams). The animals were observed at regular intervals during the course of the first day, and daily for two weeks following the treatment. The LD₅₀ (dose that causes the death of 50% of the animals) was evaluated and demonstrated the low toxicity of the compounds of the invention.

### Example B

### Effectiveness in Genetic Models

Mutations in laboratory animals and also different sensitivities to dietary regimens have allowed the development of animal models having non-insulin-dependent diabetes and hyperlipidaemia associated with obesity and with resistance to insulin.

Genetic mice models (ob/ob) (Diabetes, 1982, 31 (1), 1-6) and Zucker (fa/fa) rats have been developed by various laboratories in order to understand the physiopathology of those diseases and test the effectiveness of new antidiabetic compounds (Diabetes, 1983, 32, 830-838).

Antidiabetic and Hypolipaemic Effect in the ob/ob Mouse

The 10-week-old female ob/ob mouse (Harlan) is used for the in vivo tests. The animals are kept in a light-darkness cycle of 12 hours at 25° C. The mouse has a basal hyperglycaemia of 2 g/l. The animals are randomly selected with regard to their glycaemia to form groups of six. The compounds tested by the intraperitoneal route are dissolved in a mixture of dimethyl sulphoxide (10%) and Solutol (15%) to be administered at 10 mg/kg in a volume of 2.5 ml/kg twice per day for four days. By the per os route, the compounds are tested at 30 mg/kg administered in a volume of 2.5 ml/kg of 1% HEC twice per day for four days. The control groups receive the solvents under the same conditions as the treated groups.

The activity of the products is evaluated by measuring glycaemia 24 hours after the final administration and by measuring body weight daily.

The compounds of the invention demonstrate a very good capacity to lower glycaemia, insulinaemia and triglyderidaemia that is comparable to the effects obtained with rosiglitazone, which is used as reference substance, but with an insignificant variation in body weight. In addition, no side effects were observed during the in vivo tests.

By way of example, following treatment at a dose of 3 mg/kg for 4 days, 3-{4-[2-(6-(cyclohexylcarbonyl)-4,4-dimethyl-3,4-dihydro-1(2H)-quinolinyl)ethoxy]phenyl}-2-ethoxypropanoic acid exhibits a 57% reduction in triglyceridaemia, a 32% reduction in glycaemia and a 56% reduction in insulinaemia.

### Example C

### Pharmaceutical Composition

| | |
|---|---|
| 1000 tablets each containing a dose of 5 mg of (2S)-2-ethoxy-3-{4- | 5 g |
| [2-(6-[(methoxyimino)-(phenyl)methyl]-4,4-dimethyl-3,4-dihydro- | |
| 1(2H)-quinolinyl)ethoxy]phenyl}propanoic acid (Example 5) | |
| wheat starch | 20 g |
| maize starch | 20 g |
| lactose | 30 g |
| magnesium stearate | 2 g |
| silica | 1 g |
| hydroxypropyl cellulose | 2 g |

### Example D

### Pharmaceutical Composition

| | |
|---|---|
| 1000 tablets each containing a dose of 5 mg of 3-{4-[2-(6- | 5 g |
| (cyclohexylcarbonyl)-4,4-dimethyl-3,4-dihydro-1(2H)- | |
| quinolinyl)ethoxy]phenyl}-2-ethoxypropanoic acid | |
| (Preparation 12) | |
| wheat starch | 20 g |
| maize starch | 20 g |
| lactose | 30 g |
| magnesium stearate | 2 g |
| silica | 1 g |
| hydroxypropyl cellulose | 2 g |

### Example E

### Variation in Body Weight

Male C57 Black 6 ob/ob mice 8 to 12 weeks old were used. After being placed in quarantine for one week, they were weighed and then selected randomly with regard to their weight, and 6 homogeneous groups (starting weight not significantly different) were formed. After having been weighed, the different associations to be tested are injected intraperitoneally once per day for 7 days. The molecules are injected in a 5% DMSO/15% Solutol/q.s. H₂O solution heated to 65° C. to ensure good dissolution. The solution is in addition preheated prior to injection. The mice are weighed every day and the weight attained after 7 days of treatment is recorded.

The results obtained clearly demonstrate:
that the association according to the invention between a compound of formula (I) or of formula (V) and an antioxidant agent allows a significant reduction in the weight of the obese mice,
that there is a synergy between the 2 components of the association, the loss in weight ascertained being far greater using the association than when using each component administered on its own.

### Example F

### Pharmaceutical Composition

100 tablets each containing a dose of 30 mg of (2S)-2-ethoxy-3-{4-
[2-(6-[(methoxyimino)-(phenyl)methyl]-4,4-dimethyl-3,4-dihydro-
1(2H)-quinolinyl)ethoxy]phenyl}propanoic
acid and 10 mg of coenzyme Q₁₀

| | |
|---|---|
| (2S)-2-ethoxy-3-{4-[2-(6-[(methoxyimino)(phenyl)methyl]- | 3 g |
| 4,4-dimethyl-3,4-dihydro-1(2H)-quinolinyl)ethoxy]phenyl}- | |
| propanoic acid (Example 5) | |
| coenzyme Q₁₀ | 1 g |
| wheat starch | 20 g |
| maize starch | 20 g |
| lactose | 30 g |
| magnesium stearate | 2 g |
| silica | 1 g |
| hydroxypropyl cellulose | 2 g |

### Example G

### Pharmaceutical Composition

100 tablets each containing a dose of 30 mg of 3-{4-[2-(6-
(cyclohexylcarbonyl)-4,4-dimethyl-3,4-dihydro-1(2H)-quinolinyl)-
ethoxy]phenyl}-2-ethoxypropanoic acid

| | |
|---|---|
| 3-{4-[2-(6-(cyclohexylcarbonyl)-4,4-dimethyl-3,4-dihydro-1(2H)- | 3 g |
| quinolinyl-ethoxy]phenyl}-2-ethoxypropanoic acid | |
| (Preparation 12) | |
| coenzyme Q₁₀ | 1 g |
| wheat starch | 20 g |
| maize starch | 20 g |
| lactose | 30 g |
| magnesium stearate | 2 g |
| silica | 1 g |
| hydroxypropyl cellulose | 2 g |

1-34. (canceled)

35. A compound selected from those of formula (I): 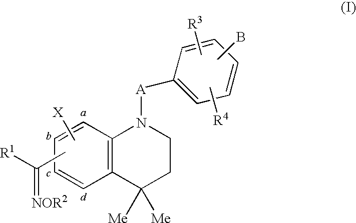
wherein:
A represents a (C₁-C₆)alkylene chain in which a CH₂ group may be replaced by a hetero atom selected from oxygen and sulphur, or by an NRₐ group (wherein Rₐ represents a hydrogen atom or a linear or branched (C₁-C₆)alkyl group), or by a phenylene or naphthylene group,
R¹ and R², which may be identical or different, each represents a hydrogen atom, a linear or branched (C₁-C₆)alkyl group, a linear or branched (C₂-C₆)alkenyl group, a linear or branched (C₂-C₆)alkynyl group, an aryl group, an aryl-(C₁-C₆)alkyl group in which the alkyl moiety may be linear or branched, an aryl-(C₂-C₆)alkenyl group in which the alkenyl moiety may be linear or branched, an aryl-(C₂-C₆)alkynyl group in which the alkynyl moiety may be linear or branched, a heteroaryl group, a heteroaryl-(C₁-C₆)alkyl group in which the alkyl moiety may be linear or branched, a heteroaryl-(C₂-C₆)alkenyl group in which the alkenyl moiety may be linear or branched, a heteroaryl-(C₂-C₆)alkynyl group in which the alkynyl moiety may be linear or branched, a (C₃-C₈)cycloalkyl group, a (C₃-C₈)cycloalkyl-(C₁-C₆)alkyl group in which the alkyl moiety may be linear or branched or a linear or branched (C₁-C₆)poly-haloalkyl group,
R³ and R⁴, which may be identical or different, each represents a hydrogen atom, a halogen atom, or an R, OR or NRR′ group wherein R and R′, which may be identical or different, each represents a hydrogen atom or a linear or branched (C₁-C₆)alkyl group, a linear or branched (C₂-C₆)alkenyl group, a linear or branched (C₂-C₆)alkynyl group, an aryl group, an aryl-(C₁-C₆)alkyl group in which the alkyl moiety may be linear or branched, an aryl-(C₂-C₆)alkenyl group in which the alkenyl moiety may be linear or branched, an aryl-(C₂-C₆)alkynyl group in which the alkynyl moiety may be linear or branched, a heteroaryl group, a heteroaryl-(C₁-C₆)alkyl group in which the alkyl moiety may be linear or branched, a heteroaryl-(C₂-C₆)alkenyl group in which the alkenyl moiety may be linear or branched, a heteroaryl-(C₂-C₆)alkynyl group in which the alkynyl moiety may be linear or branched, a (C₃-C₈)cycloalkyl group, a (C₃-C₈)cycloalkyl-(C₁-C₆)alkyl group in which the alkyl moiety may be linear or branched or a linear or branched (C₁-C₆)polyhaloalkyl group,
or R³ and R⁴, together with the carbon atoms carrying them, when they are carried by two adjacent carbon atoms, form a ring that comprises 5 or 6 ring members, wherein the ring may optionally contain a hetero atom selected from oxygen, sulphur and nitrogen,
X represents a hydrogen atom, a halogen atom, or a linear or branched (C₁-C₆)alkyl group,
B represents a linear or branched (C₁-C₆)alkyl group or a linear or branched (C₂-C₆)alkenyl group, those groups being substituted:
by a group of formula (II): 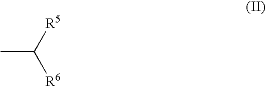
wherein 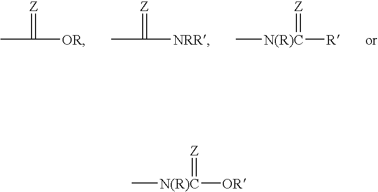
R⁵ represents a
group wherein Z represents an oxygen atom or a sulphur atom,
and R⁶ represents an aryl group, an arylalkyl group in which the alkyl moiety contains from 1 to 6 carbon atoms and may be linear or branched, a heteroaryl group, a heteroarylalkyl group in which the alkyl moiety contains from 1 to 6 carbon atoms and may be linear or branched, CN, tetrazole 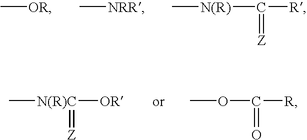
or by a group R⁷, wherein R⁷ represents a CN, tetrazole, 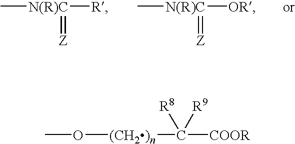
group wherein n represents 0, 1, 2, 3, 4, 5 or 6, and R⁸ and R⁹, which may be identical or different, each represents a hydrogen atom or a linear or branched (C₁-C₆)alkyl group, wherein R⁸ and R⁹ cannot simultaneously represent a hydrogen atom,
or B represents a group of formula (II) or a group R⁷,
it being understood that:
the oxime R¹—C(═N—OR²)— may have the Z or E configuration,
aryl means a phenyl, naphthyl or biphenyl group, which groups may optionally be partially hydrogenated,
heteroaryl means an aromatic mono- or bi-cyclic group containing from 5 to 10 ring members, wherein the bicyclic heteroaryl groups may optionally be partially hydrogenated on one of the rings, and wherein each ring contains from 1 to 3 hetero atoms selected from oxygen, nitrogen and sulphur,
wherein the aryl and heteroaryl groups may optionally be substituted by from 1 to 3 identical or different groups selected from linear or branched (C₁-C₆)alkyl, linear or branched (C₁-C₆)polyhaloalkyl, linear or branched (C₁-C₆)alkoxy, hydroxy, carboxy, linear or branched (C₁-C₆)alkoxycarbonyl, linear or branched (C₁-C₆)acyloxy, formyl, linear or branched (C₁-C₆)acyl, aroyl, NR_{b}R_{c} (wherein R_{b} and R_{c}, which may be identical or different, each represents a hydrogen atom, a linear or branched (C₁-C₆)alkyl group, an aryl group or a heteroaryl group), amido, nitro, cyano, and halogen atoms,
its enantiomers and diastereoisomers, and pharmaceutically acceptable addition salts thereof with an acid or a base.

36. The compound of claim 35, wherein the group R¹—C(═N—OR²)— is in the c position.

37. The compound of claim 35, wherein R³ and R⁴ represent a hydrogen atom.

38. The compound of claim 35, wherein X represents a hydrogen atom.

39. The compound of claim 35, wherein X represents an alkyl group.

40. The compound of claim 35, wherein A represents an ethyleneoxy group.

41. The compound of claim 35, wherein R² represents a hydrogen atom.

42. The compound of claim 35, wherein R² represents an alkyl group.

43. The compound of claim 35, wherein R¹ represents an unsubstituted or substituted phenyl group.

44. The compound of claim 35, wherein B represents an alkyl group substituted by a 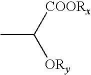
group wherein Rₓ and R_{y}, which may be identical or different, each represents a hydrogen atom or an alkyl group.

45. The compound of claim 35, wherein B represents a 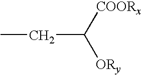
group wherein Rₓ and R_{y}, which may be identical or different, each represents a hydrogen atom or an alkyl group.

46. The compound of claim 35, which is selected from:
ethyl 2-ethoxy-3-{4-[2-(6-[(hydroxyimino)(phenyl)methyl]-4,4-dimethyl-3,4-dihydro-1(2H)-quinolinyl)ethoxy]phenyl}propanoate,
ethyl 2-ethoxy-3-{4-[2-(6-[(methoxyimino)(phenyl)methyl]-4,4-dimethyl-3,4-dihydro-1(2H)-quinolinyl)ethoxy]phenyl}propanoate,
2-ethoxy-3-{4-[2-(6-[(methoxyimino)(phenyl)methyl]-4,4-dimethyl-3,4-dihydro-1(2H)-quinolinyl)ethoxy]phenyl}propanoic acid,
ethyl (2S)-2-ethoxy-3-{4-[2-(6-[(methoxyimino)(phenyl)methyl]-4,4-dimethyl-3,4-dihydro-1(2H)-quinolinyl)ethoxy]phenyl}propanoate,
(2S)-2-ethoxy-3-{4-[2-(6-[(methoxyimino)(phenyl)methyl]-4,4-dimethyl-3,4-dihydro-1(2H)-quinolinyl)ethoxy]phenyl}propanoic acid,
ethyl 3-{4-[2-(6-[(methoxyimino)(phenyl)methyl]-4,4-dimethyl-3,4-dihydro-1(2H)-quinolinyl)ethoxy]phenyl}-2-[4-(trifluoromethyl)phenoxy]propanoate,
2-(acetoxy)-3-{4-[2-(6-[(methoxyimino)(phenyl)methyl]-4,4-dimethyl-3,4-dihydro-1(2H)-quinolinyl)ethoxy]phenyl}propanoic acid,
ethyl 3-{4-[2-(6-[(methoxyimino)(phenyl)methyl]-4,4-dimethyl-3,4-dihydro-1(2H)-quinolinyl)ethoxy]phenyl}-2-(2,2,2-trifluoroethoxy)propanoate,
3-{4-[2-(6-[(methoxyimino)(phenyl)methyl]-4,4-dimethyl-3,4-dihydro-1(2H)-quinolinyl)ethoxy]phenyl}-2-(2,2,2-trifluoroethoxy)propanoic acid,
ethyl 2-(2-benzoylanilino)-3-{4-[2-(6-[(methoxyimino)(phenyl)methyl]-4,4-dimethyl-3,4-dihydro-1(2H)-quinolinyl)ethoxy]phenyl}propanoate,
2-(2-benzoylanilino)-3-{4-[2-(6-[(methoxyimino)(phenyl)methyl]-4,4-dimethyl-3,4-dihydro-1(2H)-quinolinyl)ethoxy]phenyl}propanoic acid,
ethyl (2S)-3-{4-[2-(6-[(4-cyanophenyl)(methoxyimino)methyl]-4,4-dimethyl-3,4-dihydro-1(2H)-quinolinyl)ethoxy]phenyl}-2-ethoxypropanoate,
ethyl 4-[[1-(2-{4-[(2S)-2,3-diethoxy-3-oxopropyl]phenoxy}ethyl)-4,4-dimethyl-1,2,3,4-tetrahydro-6-quinolinyl](methoxyimino)methyl]benzoate,
4-[[1-(2-{4-[(2S)-2-carboxy-2-ethoxyethyl]phenoxy}ethyl)-4,4-dimethyl-1,2,3,4-tetrahydro-6-quinolinyl](methoxyimino)methyl]benzoic acid,
ethyl 2-ethoxy-3-{4-[2-(6-[(hydroxyimino)(phenyl)methyl]-4,4,7-trimethyl-3,4-dihydro-1(2H)-quinolinyl)ethoxy]phenyl}propanoate,
2-ethoxy-3-{4-[2-(6-[(hydroxyimino)(phenyl)methyl]-4,4,7-trimethyl-3,4-dihydro-1(2H)-quinolinyl)ethoxy]phenyl}propanoic acid,
ethyl 3-{4-[2-(6-[cyclohexyl(hydroxyimino)methyl]-4,4-dimethyl-3,4-dihydro-1(2H)-quinolinyl)ethoxy]phenyl}-2(S)-ethoxypropanoate, 3-{4-[2-(6-[cyclohexyl(hydroxy-imino)methyl]-4,4-dimethyl-3,4-dihydro-1(2H)-quinolinyl)ethoxy]phenyl}-2(S)-ethoxypropanoic acid,
enantiomers and diastereoisomers thereof, and pharmaceutically acceptable addition salts thereof with an acid or a base.

47. A compound selected from those of formula (V): 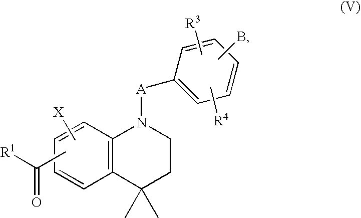
wherein,
A represents a (C₁-C₆)alkylene chain in which a CH₂ group may be replaced by a hetero atom selected from oxygen and sulphur, or by an NRₐ group (wherein R₁ represents a hydrogen atom or a linear or branched (C₁-C₆)alkyl group), or by a phenylene or naphthylene group,
R¹ represents a hydrogen atom, a linear or branched (C₁-C₆)alkyl group, a linear or branched (C₂-C₆)alkenyl group, a linear or branched (C₂-C₆)alkynyl group, an aryl group, an aryl-(C₁-C₆)alkyl group in which the alkyl moiety may be linear or branched, an aryl-(C₂-C₆)alkenyl group in which the alkenyl moiety may be linear or branched, an aryl-(C₂-C₆)alkynyl group in which the alkynyl moiety may be linear or branched, a heteroaryl group, a heteroaryl-(C₁-C₆)alkyl group in which the alkyl moiety may be linear or branched, a heteroaryl-(C₂-C₆)alkenyl group in which the alkenyl moiety may be linear or branched, a heteroaryl-(C₂-C₆)alkynyl group in which the alkynyl moiety may be linear or branched, a (C₃-C₈)cycloalkyl group, a (C₃-C₈)cycloalkyl-(C₁-C₆)alkyl group in which the alkyl moiety may be linear or branched or a linear or branched (C₁-C₆)polyhaloalkyl group,
R³ and R⁴, which may be identical or different, each represents a hydrogen atom, a halogen atom, or an R, OR or NRR′ group wherein R and R′, which may be identical or different, each represents a hydrogen atom or a linear or branched (C₁-C₆)alkyl group, a linear or branched (C₂-C₆)alkenyl group, a linear or branched (C₂-C₆)alkynyl group, an aryl group, an aryl-(C₁-C₆)alkyl group in which the alkyl moiety may be linear or branched, an aryl-(C₂-C₆)alkenyl group in which the alkenyl moiety may be linear or branched, an aryl-(C₂-C₆)alkynyl group in which the alkynyl moiety may be linear or branched, a heteroaryl group, a heteroaryl-(C₁-C₆)alkyl group in which the alkyl moiety may be linear or branched, a heteroaryl-(C₂-C₆)alkenyl group in which the alkenyl moiety may be linear or branched, a heteroaryl-(C₂-C₆)alkynyl group in which the alkynyl moiety may be linear or branched, a (C₃-C₈)cycloalkyl group, a (C₃-C₈)cycloalkyl-(C₁-C₆)alkyl group in which the alkyl moiety may be linear or branched or a linear or branched (C₁-C₆)polyhaloalkyl group,
or R³ and R⁴, together with the carbon atoms carrying them, when they are carried by two adjacent carbon atoms, form a ring that comprises 5 or 6 ring members, wherein the ring may optionally contain a hetero atom selected from oxygen, sulphur and nitrogen,
X represents a hydrogen atom, a halogen atom, or a linear or branched (C₁-C₆)alkyl group,
B represents a linear or branched (C₁-C₆)alkyl group or a linear or branched (C₂-C₆)alkenyl group, those groups being substituted:
by a group of formula (II): 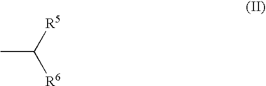
wherein: 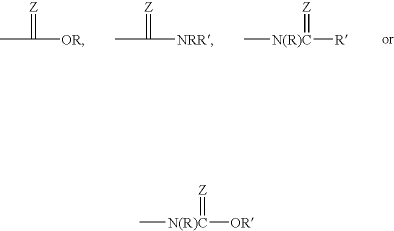
R⁵ represents a
group wherein Z represents an oxygen atom or a sulphur atom,
and R⁶ represents an aryl group, an arylalkyl group in which the alkyl moiety contains from 1 to 6 carbon atoms and may be linear or branched, a heteroaryl group, a heteroarylalkyl group in which the alkyl moiety contains from 1 to 6 carbon atoms and may be linear or branched, CN, tetrazole 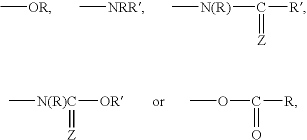
or by a group R⁷, wherein R⁷ represents a CN, tetrazole, 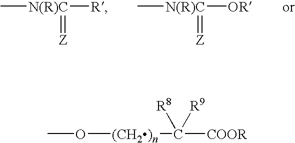
group wherein n represents 0, 1, 2, 3, 4, 5 or 6, and R⁸ and R⁹, which may be identical or different, each represents a hydrogen atom or a linear or branched (C₁-C₆)alkyl group, wherein R⁸ and R⁹ cannot simultaneously represent a hydrogen atom,
or B represents a group of formula (II) or a group R⁷,
it being understood that:
aryl means a phenyl, naphthyl or biphenyl group, which groups may optionally be partially hydrogenated,
heteroaryl means an aromatic mono- or bi-cyclic group containing from 5 to 10 ring members, wherein the bicyclic heteroaryl groups may optionally be partially hydrogenated on one of the rings, and wherein each ring contains from 1 to 3 hetero atoms selected from oxygen, nitrogen and sulphur,
wherein the aryl and heteroaryl groups may optionally be substituted by from 1 to 3 identical or different groups selected from linear or branched (C₁-C₆)alkyl, linear or branched (C₁-C₆)polyhaloalkyl, linear or branched (C₁-C₆)alkoxy, hydroxy, carboxy, linear or branched (C₁-C₆)alkoxycarbonyl, linear or branched (C₁-C₆)acyloxy, formyl, linear or branched (C₁-C₆)acyl, aroyl, NR_{b}R_{c}(wherein R_{b} and R_{c}, which may be identical or different, each represents a hydrogen atom, a linear or branched (C₁-C₆)alkyl group, an aryl group or a heteroaryl group), amido, nitro, cyano, and halogen atoms,
its enantiomers and diastereoisomers, and pharmaceutically acceptable addition salts thereof with an acid or a base,
for use as an intermediate in the synthesis of the compounds of formula (I) and as a hypoglycaemic and hypolipaemic agent.

48. A pharmaceutical composition comprising as active ingredient at least one compound of claim 35, or a pharmaceutically acceptable addition salt thereof with an acid or a base, alone or in combination with one or more pharmaceutically acceptable excipients.

49. A pharmaceutical composition comprising as active ingredient at least one compound of claim 47, or a pharmaceutically acceptable addition salt thereof with an acid or a base, alone or in combination with one or more pharmaceutically acceptable excipients.

50. A method of treating a living animal body, including a human, afflicted with a condition selected from hyperglycaemia, dyslipidaemia, non-insulin-dependent type II diabetes, insulin resistance, glucose intolerance, disorders associated with syndrome X, coronary artery disease, cardiovascular diseases, renal disorders, retinopathy, disorders associated with the activation of endothelial cells, psoriasis, polycystic ovary syndrome, dementia, osteoporosis, intestinal inflammatory disorders, myotonic dystrophy, pancreatitis, arteriosclerosis, xanthoma, type I diabetes, obesity, conditions requiring regulation of appetite, anorexia, bulimia, anorexia nervosa, cancer, and conditions requiring an angiogenesis inhibitor, comprising the step of administering to the living animal body, including a human, an amount of a compound of claim 35 which is effective for treatment of the condition.

51. The method of claim 50, wherein the cancer is selected from hormone-dependent cancer, breast cancer and colon cancer.

52. A method of treating a living animal body, including a human, afflicted with a condition selected from hyperglycaemia, dyslipidaemia, non-insulin-dependent type II diabetes, insulin resistance, glucose intolerance, disorders associated with syndrome X, coronary artery disease, cardiovascular diseases, renal disorders, retinopathy, disorders associated with the activation of endothelial cells, psoriasis, polycystic ovary syndrome, dementia, osteoporosis, intestinal inflammatory disorders, myotonic dystrophy, pancreatitis, arteriosclerosis, xanthoma, type I diabetes, obesity, conditions requiring regulation of appetite, anorexia, bulimia, anorexia nervosa, cancer, and conditions requiring an angiogenesis inhibitor, comprising the step of administering to the living animal body, including a human, an amount of a compound of claim 47 which is effective for treatment of the condition.

53. The method of claim 52, wherein the cancer is selected from hormone-dependent cancer, breast cancer and colon cancer.

54. A composition comprising a combination of the compound of claim 35 and an antioxidant agent.

55. The composition of claim 54, wherein the compound of claim 35 is (2S)-2-ethoxy-3-{4-[2-(6-[(methoxyimino)(phenyl)methyl]-4,4-dimethyl-3,4-dihydro-1(2H)-quinolinyl)ethoxy]phenyl}propanoic acid, an enantiomer or diastereoisomer thereof or a pharmaceutically acceptable addition salt thereof with an acid or a base.

56. The composition of claim 54, wherein the antioxidant agent is coenzyme Q₁₀.

57. The composition of claim 54, wherein the antioxidant agent is vitamin E.

58. The composition of claim 54, which is (2S)-2-ethoxy-3-{4-[2-(6-[(methoxy-imino)(phenyl)methyl]-4,4-dimethyl-3,4-dihydro-1(2H)-quinolinyl)ethoxy]phenyl}-propanoic acid and coenzyme Q₁₀.

59. The composition of claim 54, further comprising one or more pharmaceutically acceptable excipients.

60. A method of treating a living animal body, including a human, afflicted with obesity, comprising the step of administering to the living animal body, including a human, an amount of a composition of claim 54, which is effective for treatment of obesity.

61. A method of treating a living animal body, including a human, afflicted with obesity caused by a therapeutic treatment, comprising the step of administering to the living animal body, including a human, an amount of a composition of claim 54, which is effective for treatment of obesity caused by a therapeutic treatment.

62. A method of treating a living animal body, including a human, afflicted with obesity caused by a treatment of type I or type II diabetes, comprising the step of administering to the living animal body, including a human, an amount of a composition of claim 54, which is effective for treatment of obesity caused by treatment of type I or type II diabetes.

63. A method of treating a living animal body, including a human, afflicted with overweight characterised by a body mass index greater than 25 and less than 30, comprising the step of administering to the living animal body, including a human, an amount of a composition of claim 54, which is effective for treatment of overweight characterised by a body mass index greater than 25 and less than 30.

64. A method of treating a living animal body, including a human, afflicted with overweight characterised by a body mass index greater than 25 and less than 30 caused by a therapeutic treatment, comprising the step of administering to the living animal body, including a human, an amount of a composition of claim 54, which is effective for treatment of overweight characterised by a body mass index greater than 25 and less than 30 caused by a therapeutic treatment.

65. A method of treating a living animal body, including a human, afflicted with overweight characterised by a body mass index greater than 25 and less than 30 caused by a treatment of type I or type II diabetes, comprising the step of administering to the living animal body, including a human, an amount of a composition of claim 54, which is effective for treatment of overweight characterised by a body mass index greater than 25 and less than 30 caused by a treatment of type I or type II diabetes.

66. A composition comprising a combination of the compound of claim 47 and an antioxidant agent.

67. The composition of claim 66, wherein the compound of claim 47 is 3-{4-[2-(6-(cyclohexylcarbonyl)-4,4-dimethyl-3,4-dihydro-1(2H)-quinolinyl)ethoxy]phenyl}-2-ethoxypropanoic acid, an enantiomer or diastereoisomer thereof, or a pharmaceutically acceptable addition salt thereof with an acid or a base.

68. The composition of claim 66, wherein the antioxidant agent is coenzyme Q₁₀.

69. The composition of claim 66, wherein the antioxidant agent is vitamin E.

70. The composition of claim 66, which is 3-{4-[2-(6-(cyclohexylcarbonyl)-4,4-dimethyl-3,4-dihydro-1(2H)-quinolinyl)ethoxy]phenyl}-2-ethoxypropanoic acid and coenzyme Qlo.

71. The composition of claim 66, further comprising one or more pharmaceutically acceptable excipients.

72. A method of treating a living animal body, including a human, afflicted with obesity, comprising the step of administering to the living animal body, including a human, an amount of a composition of claim 66, which is effective for treatment of obesity.

73. A method of treating a living animal body, including a human, afflicted with obesity caused by a therapeutic treatment, comprising the step of administering to the living animal body, including a human, an amount of a composition of claim 66, which is effective for treatment of obesity caused by a therapeutic treatment.

74. A method of treating a living animal body, including a human, afflicted with obesity caused by a treatment of type I or type II diabetes, comprising the step of administering to the living animal body, including a human, an amount of a composition of claim 66, which is effective for treatment of obesity caused by treatment of type I or type II diabetes.

75. A method of treating a living animal body, including a human, afflicted with overweight characterised by a body mass index greater than 25 and less than 30, comprising the step of administering to the living animal body, including a human, an amount of a composition of claim 66, which is effective for treatment of overweight characterised by a body mass index greater than 25 and less than 30.

76. A method of treating a living animal body, including a human, afflicted with overweight characterised by a body mass index greater than 25 and less than 30 caused by a therapeutic treatment, comprising the step of administering to the living animal body, including a human, an amount of a composition of claim 66, which is effective for treatment of overweight characterised by a body mass index greater than 25 and less than 30 caused by a therapeutic treatment.

77. A method of treating a living animal body, including a human, afflicted with overweight characterised by a body mass index greater than 25 and less than 30 caused by a treatment of type I or type II diabetes, comprising the step of administering to the living animal body, including a human, an amount of a composition of claim 66, which is effective for treatment of overweight characterised by a body mass index greater than 25 and less than 30 caused by a treatment of type I or type II diabetes.
